(19) United States
(12) Patent Application Publication
(10) Pub. No.: US 2003/0028046 A1
(43) Pub. Date: Feb. 6, 2003

(54) Method for producing chlorocarboxylic acid chlorides

(76) Inventors: Armin Stamm, Nieder-Olm (DE); Roland Gotz, Neulussheim (DE); Jochem Henkelmann, Mannheim (DE); Friedrich Closs, Mannheim (DE); Heinz-Josef Kneuper, Niederkirchen (DE)

Correspondence Address: KEIL & WEINKAUF, 1350 CONNECTICUT AVENUE, N.W., WASHINGTON, DC, 20036, US

(21) Appl. No.: 10/220,155

(22) PCT Filed: Feb. 28, 2001
(86) PCT No.: PCT/EP01/02238

(51) Int. Cl. C07C 51/58

(52) U.S. Cl. 558/440; 562/857

(57) ABSTRACT

A process for the preparation of chlorocarboxylic chlorides of formula (I)

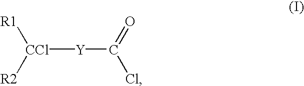

in which

R¹ and R² independently denote

a hydrogen atom, a carbon-containing organic radical, a halogen, or a nitro or cyano group;

and Y denotes

an alkylene chain which contains from 1 to 10 carbons in the chain and which is unsubstituted or substituted by carbon-containing organic radicals, halogen, nitro and/or cyano groups, and the alkylene chain can be interrupted by an ether, thioether, tertiary amino or keto group,

and the carbon-containing organic radicals of Y and/or R¹ and/or R² can be bonded to each other so as to form a non-aromatic system,

by reaction of a lactone of formula (II)

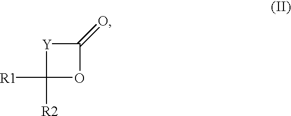

in which R¹, R² and Y have the meanings stated above, with a chlorinating agent in the presence of a chlorinating catalyst, in which the reaction is carried out in the presence of a boron compound.

### DESCRIPTION

The present invention relates to a process for the preparation of chlorocarboxylic chlorides of formula (I)

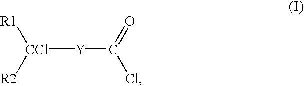

in which

R¹ and R² independently denote

a hydrogen atom, a carbon-containing organic radical, a halogen, or a nitro or cyano group;

and Y denotes

an alkylene chain which contains from 1 to 10 carbons in the chain and which is unsubstituted or substituted by carbon-containing organic radicals, halogen, nitro and/or cyano groups, and the alkylene chain can be interrupted by an ether, thioether, tertiary amino or keto group,

and the carbon-containing organic radicals of Y and/or R¹ and/or R² can be bonded to each other so as to form a non-aromatic system,

by reaction of a lactone of formula (II)

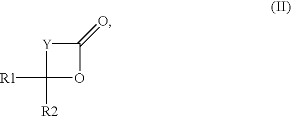

in which R¹ and R² and Y have the meanings stated above, with a chlorinating agent in the presence of a chlorinating catalyst.

Chlorocarboxylic chlorides are important reactive intermediate products for the preparation of pharmaceutical and agrochemical active substances.

Chlorocarboxylic chlorides can be prepared, for example, by reaction of the corresponding lactones with chlorinating agents in the presence of a catalyst. The chlorinating agents used are typically phosgene or thionyl chloride, since they form, as coupling products, exclusively gaseous substances (CO₂ or SO₂ and HCl).

When use is made of thionyl chloride as chlorinating agent zinc chloride is usually employed as catalyst. Appropriate processes are described in I. I. Grandberg et.al, Izv. Timiryazevsk. S.kh. Akad. 1974, (6), pages 198 to 204 and O. P. Goel et. al., Synthesis, 1973, pages 538 to 539. The conversion of γ-butyrolactone to 4-chlorobutyric chloride gave yields of from 65 to 80%.

When use is made of phosgene as chlorinating agent various catalyst systems are generally used. U.S. Pat. No. 2,778,852 mentions the following as being suitable catalysts: pyridines, tertiary amines, heavy metals and acids, such as sulfuric acid, phosphoric acid, phosphorus chloride, phosphorus oxychloride, aluminum chloride, sulfuryl chloride and chlorosulfuric acid. Suitable catalysts are, according to laid-open specification DE-A 19,753,773, urea compounds, according to laid-open specifications EP-A 0,413,264 and EP-A 0,435,714, phosphine oxides, and according to laid-open specifications EP-A 0,253,214 and EP-A 0,583,589, organonitrogen compounds such as quaternary ammonium salts, heterocyclic nitrogen compounds, amines or formamides.

U.S. Pat. No. 2,778,852 describes the synthesis of 4-chlorobutyric chloride by reaction of γ-butyrolactone with phosgene in the presence of pyridine.

In order to increase the yield, hydrogen chloride gas is usually additionally introduced. The use of hydrogen chloride is however disadvantageous, particularly for ecological and economical reasons, since it is used in hyperstoichiometric amounts and the excess portion must be purified and neutralized, which leads to considerable accumulation of salt. Furthermore, the use of large amounts of hydrogen chloride gas must meet additional technological and logistic requirements.

The object of the present invention is thus to provide a process for the preparation of chlorocarboxylic chlorides by reaction of the corresponding lactones with chlorinating agents, which process no longer suffers from the known drawbacks and produces the chlorocarboxylic chlorides in a high yield and high state of purity.

Accordingly, we have found a process for the preparation of chlorocarboxylic chlorides of formula (I)

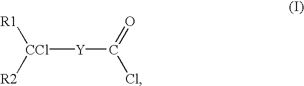

in which

R¹ and R² independently denote

a hydrogen atom, a carbon-containing organic radical, a halogen, or a nitro or cyano group;

and Y denotes

an alkylene chain which contains from 1 to 10 carbons in the chain and which is unsubstituted or substituted by carbon-containing organic radicals, halogen, nitro and/or cyano groups, and the alkylene chain can be interrupted by an ether, thioether, tertiary amino or keto group,

and the carbon-containing organic radicals of Y and/or R¹ and/or R² can be bonded to each other so as to form a non-aromatic system,

by reaction of a lactone of formula (II)

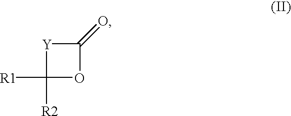

in which R¹, R² and Y have the meanings stated above, with a chlorinating agent in the presence of a chlorinating catalyst, which is characterized in that conversion is carried out in the presence of a boron compound.

An essential feature of the process of the invention is the presence of a boron compound. Examples of suitable boron compounds are the compounds and groups of substances listed below, mixtures of different boron compounds being likewise possible:

boron oxide, such as B₂O₃;

boric oxy acids, such as boric acid (H₃BO₃, more correctly: “orthoboric acid”), metaboric acids (of the type HBO₂, eg α-HBO₂, β-HBO₂ or γ-HBO₂), oligoboric acids or polyboric acids;

salts of boric oxy acids, such as borates ([BO₃]³−, more correctly: “orthoborate”), oligoborates (eg [B₃O₃ (OH)₅]²⁻, [B₄O₅(OH)₄]²⁻, [B₅O₆(OH)₆]³⁻ or [B₆O₇(OH)₆]²⁻) or polyborates (eg [BO₂]⁻) with inorganic or organic cations, for example alkali metal ions (eg Li⁺, Na⁺ or K⁺), alkaline earth metal ions (eg Mg²⁺, Ca²⁺ or Sr²⁺), the ammonium ion NH₄⁺ or primary, secondary, tertiary or quaternary amines (eg tetramethylammonium, tetraethylammonium, tetrapropylammonium, tetraisopropylammonium, phenyltrimethylammonium, phenyltriethylammonium, trimethylammonium, triethylammonium, tripropylammonium, triisopropylammonium, phenyldimethylammonium, phenyldiethylammonium or phenylammonium (“anilinium”));

boronic acids (R—B(OH)₂) and their inorganic or organic salts, such as benzeneboronic acid (dihydroxyphenylborane) or disodium phenyl boronate;

boric acid esters, such as the mono-, di- or tri-(C₁-C₆ alkyl) esters having the same or different, unbranched or branched alkyl groups (eg methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-l-methylpropyl or 1-ethyl-2-methylpropyl), for example trimethyl borate, triethyl borate or tripropyl borate;

boron halides containing fluorine, chlorine, bromine and/or iodine, for example BF₃ (boron trifluoride), BC1₃ (boron trichloride), BBr₃ (boron tribromide), BI₃ (boron triiodide), BF₂Cl, BFCl₂, BF₂Br, BFBr₂, BF₂I, BFI₂, BFClBr, BFClI, BFBrI, BCl₂Br, BClBr₂, BCl₂I, BClI₂, BClBrI, BBr₂I, BBrI₂, B₂F₄, B₂Cl₄, B₂Br₄, B₂I₄ and their complexes, for example with oxygen, sulfur or nitrogen compounds, such as hydrates, alkoxides, etherates, complexes with sulfides, ammonia, amines or pyridines, for example [water.BF₃], [methanol.BF₃], [ethanol.BF₃], [dimethyl ether.BF₃], [diethyl ether.BF₃], [n-propyl ether.BF₃], [diisopropyl ether.BF₃], [tetrahydrofuran.BF₃], [dimethyl sulfide.BF₃], [ammonia.BF₃], [methylamine.BF₃], [dimethylamine.BF₃], [trimethylamine.BF₃], [ethylamine.BF₃], [diethylamine.BF₃], [triethylamine.BF₃], [urea.BF₃], [pyridine.BF₃], [2-methylpyridine.BF₃] or [3-methylpyridine.BF₃].

The compounds preferably used are

boron oxide B₂O₃;

boric acid H₃BO₃;

tri(C₃-C₄ alkyl) borates, such as trimethyl borate, triethyl borate, tripropyl borate, triisopropyl borate or tributyl borate;

boron trifluoride, boron trichloride or their complexes, for example with water, alcohols (particularly methanol), ethers (particularly diethyl ether), sulfides (particularly dimethyl sulfide) or amines (particularly ethylamine), for example boron trifluoride dihydrate or boron trifluoride etherates (particularly with diethyl ether);

or mixtures thereof.

Very preferably used are the halogen-free boron compounds boron oxide B₂O₃, boric acid H₃BO₃ and tri(C₁-C₄ alkyl) borate. Particularly preferred are boric acid H₃BO₃ and trimethyl borate. The use of such boron compounds has the advantage that the reaction mixtures are free from fluoride ions. This simplifies the entire apparatus technology as against the reaction involving boron halides.

In the process of the invention the boron compound or mixture thereof is used in a concentration of from 0.1 to 20 mol %, preferably from 0.1 to 10 mol % and more preferably from 0.5 to 5 mol % based on the lactone (II).

The chlorocarboxylic chlorides produced by the process of the invention conform to formula (I)

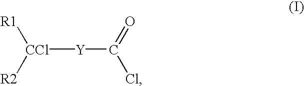

in which R¹ and R² independently denote a hydrogen atom, a carbon-containing organic radical, a halogen or a nitro or cyano group.

By a carbon-containing organic radical we mean an unsubstituted or substituted, aliphatic, aromatic or araliphatic radical containing from 1 to 20 carbons. This radical can contain one or more heteroatoms, such as oxygen, nitrogen or sulfur, for example —O—, —S—, —NR—, —CO— and/or —N═ in aliphatic or aromatic systems, and/or may be substituted by one or more functional groups containing, for example, oxygen, nitrogen, sulfur and/or halogen, for example substituted by fluorine, chlorine, bromine, iodine and/or cyano. If the carbon-containing organic radical contains one or more heteroatoms, it may be bonded via a heteroatom. Thus ether, thioether and tertiary amino groups are for example also enclosed. As preferred examples of the carbon-containing organic radical there may be mentioned C₁-C₂₀ alkyl, particularly C₁-C₆ alkyl, C₆-C₁₀ aryl, C₇-C₂₀ aralkyl, particularly C₇-C₁₀ aralkyl, and C₇-C₂₀ alkaryl, particularly C₇-C₁₀ alkaryl.

As examples of halogens there may be mentioned fluorine, chlorine, bromine and iodine.

Preference is given to chlorocarboxylic chlorides (I) in which R¹ and R² independently denote hydrogen, C₁-C₆ alkyl, C₆-C₁₀ aryl, C₇-C₁₀ aralkyl or C₇-C₁₀ alkaryl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, phenyl, 2-methylphenyl (o-toluoyl), 3-methylphenyl (m-toluoyl), 4-methylphenyl (p-toluoyl), naphthyl or benzyl. Special preference is given to hydrogen and C₁-C₄ alkyl, particularly hydrogen.

Y denotes an alkylene chain having from 1 to 10 carbons in the chain which may be unsubstituted or substituted by carbon-containing organic radicals, halogen, nitro and/or cyano groups, and the alkylene chain can be interrupted by an ether(—O—), thioether(—S—), tertiaere amino(—NR—) or keto(—CO—) group. The carbon-containing organic radicals and halogen are as defined above.

As examples of the radical Y there may be mentioned the alkenes (CH₂)ₙ, in which n is equal to from 1 to 10 and in which one or more or possibly all of the hydrogen atoms can be replaced by C₁-C₆ alkyl, C₆-C₁₀ aryl, C₇-C₁₀ aralkyl and/or C₇-C₁₀ alkaryl, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-l-methylpropyl, 1-ethyl-2-methylpropyl, phenyl, 2-methylphenyl (o-toluoyl), 3-methylphenyl (m-toluoyl), 4-methylphenyl (p-toluoyl), naphthyl or benzyl.

Preference is given to chlorocarboxylic chlorides (I) in which Y denotes an unsubstituted alkene (CH₂)ₙ in which n is equal to from 2 to 8, preferably from 2 to 4, such as CH₂CH₂, CH₂CH₂CH₂ and CH₂CH₂CH₂CH₂.

Possibly, the organic radicals R¹ and/or R² and/or those of Y are bonded to each other to form a non-aromatic system. As an example thereof there may be mentioned hexahydrophthalide.

The chlorocarboxylic chlorides (I) that are greatly preferred as products of the process of the invention are 4-chlorobutyric chloride (4-chlorobutanoic chloride), 5-chlorovaleric chloride (5-chloropentanoic chloride) or 6-chlorocaproic chloride (6-chlorohexanoic chloride).

The lactones to be used conform to formula (II)

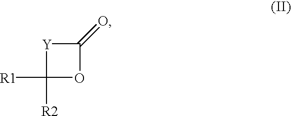

in which R¹, R² and Y have the meanings stated above. Of course, mixtures of different lactones can be used if desired. We very much prefer to use γ-butyrolactone, δ-valerolactone or ε-caprolactone.

The chlorinating agents used are preferably phosgene, diphosgene (trichloromethyl chloroformate), triphosgene (bis(trichloromethyl)carbonate) and/or thionyl chloride. Particularly preferred is the use of phosgene or thionyl chloride, particularly gaseous and/or liquid phosgene.

Suitable chlorinating catalysts are theoretically all known chlorinating catalysts, particularly nitrogen and phosphorus compounds, such as open-chain or cyclic, unsubstituted or substituted ureas, di-N,N-substituted formamides (eg N,N-dimethylformamide), trialkyl phosphine oxides or unsubstituted or substituted triarylphosphine oxides, substituted or unsubstituted pyridines, quaternary ammonium salts (eg benzyltrimethylammonium chloride), amidines or salts thereof including hydrochlorides, unsubstituted or mono- or poly-N-substituted guanidines or hexaalkylguanidinium salts.

The chlorinating catalyst used is preferably a urea compound, a phosphine oxide, a pyridine compound or a mixture thereof.

The urea compounds that are preferably used are described, for example, in laid-open specification DE-A 19,753,773. We particularly prefer to use open-chain, substituted urea compounds of formula (III)

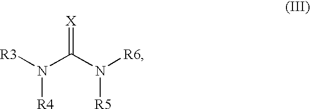

in which X stands for oxygen or sulfur and R³ to R² independently denote preferably C₁-C₁₀ alkyl or in which one of the radicals R³ or R² forms, together with one of the radicals R⁵ or R², a C₂-C₄ alkylene chain. Very special preference is given to urea compounds which are liquid under the conditions of the reaction, for example N,N′-dimethylethylene urea (1,3-dimethyl-2-imidazolidinone), N,N′-dimethylpropylene urea (1,3-dimethyltetrahydro-2(1H)-pyrimidinone), N,N,N′,N′-tetrabutyl urea or N,N,N′,N′-tetramethylthio urea. The said urea compounds can be used as such or in the form of their salts with hydrochloric acid, for example as hydrochlorides, or in the form of their Vilsmeier-type salts as can be obtained by reaction with phosgene, but the hydrochlorides are preferred.

The phosphine oxides that are preferably used are described, for example, in laid-open specification EP-A 0,413,264. We particularly prefer to use the trialkyl phosphine oxides or unsubstituted or substituted triarylphosphine oxides of formula (IV)

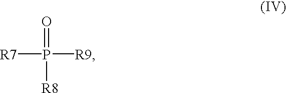

in which R⁷ to R⁹ independently denote preferably C₁-C₁₀ alkyl or unsubstituted or (C₁-C₄ alkyl)-substituted phenyl. Very special preference is given to phosphine oxides which are liquid under the conditions of the reaction, for example linear or branched trioctyl, trihexyl or tributyl phosphine oxides and also triphenylphosphine oxide or mixtures of different trialkyl phosphine oxides (eg Cyanex sold by Cytec Industries).

The substituted or unsubstituted pyridines that are preferably used are represented by formula (V)

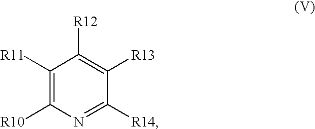

in which R¹⁰ to R¹⁴ independently denote preferably hydrogen or C₁-C₄ alkyl. Another possibility is that two adjacent radicals may be bonded to each other to form a non-aromatic or aromatic system. Special preference is given to the mono(C₁-C₄ alkyl)pyridines and most preferably the monomethylpyridines, particularly 3-methylpyridine (β-picoline).

In the process of the invention, particularly 3-methylpyridine, triphenylphosphine oxide and/or trialkyl phosphine oxide are used.

The use of liquid chlorinating catalysts has primarily process engineering advantages. For example, there is no complicated handling of solids and metering and transport thereof. Furthermore substantially less viscous bottoms are obtain in the following workup distillation stage and choking is avoided.

The chlorinating catalyst is used, in the process of the invention, in a concentration of from 0.1 to 20 mol %, preferably from 0.1 to 10 mol % and more preferably from 0.5 to 5 mol % based on the lactone (II).

In another preferred embodiment of the process, the catalyst is used in the form of a complex of the boron compound and the chlorinating catalyst. This can be prepared, for example, by admixture of the two components upstream of or in the reactor. An example of a suitable complex is the BF₃-β-picoline complex.

The reactors used for the chlorination can be, theoretically, any apparatus for vapor-liquid or liquid-liquid reactions described in the relevant technical literature. To achieve a high space-time yield, it is important to effect intense intermixture between the lactone, the solution containing the chlorinating catalyst and the boron compound, and the added chlorinating agent. As non-restrictive examples there may be mentioned agitated tanks, cascades of stirred-tank reactors, countercurrent reaction columns, flow tubes (preferably fitted with baffles), bubble columns and loop reactors.

The process is preferably carried out without the use of solvent. It is possible, however, to add a solvent that is inert to the chlorinating agent used. Inert solvents are for example aromatic hydrocarbons, such as toluene, chlorobenzene, o-, m- or p-dichlorobenzene, o-, m- or p-xylene, cyclic carbonates, such as ethylene carbonate or propylene carbonate, the same chlorocarboxylic acid chloride as that to be produced or mixtures thereof. If solvents are used, preferably the same chlorocarboxylic acid chloride as that to be produced is used. The addition of a solvent can be of advantage for example when use is made of lactones (II) that are high-molecular, have a high-viscosity or are solid under the conditions of the reaction.

The process of the invention can be carried out at a temperature of from 50° to 200° C., preferably from 80° to 200° C., and more preferably from 110° to 160° C. It is generally carried out under a pressure of from 0.01 to 5 MPa absolute, preferably under a pressure of from 0.5 to 2 MPa absolute and more preferably under atmospheric pressure.

The total amount of phosgene that is introduced in the process of the invention is generally from 0.8 to 1.5 mol and preferably from 0.9 to 1.2 mol per mol of lactone (II).

Addition of the educts (lactone (II) and chlorinating agent) and the catalysts (chlorinating catalyst and boron compound) can generally take place in any order. Preferably, in one variant, the lactone (II), the chlorinating catalyst, the boron compound and optionally a solvent are used as initial batch and the chlorinating agent is then introduced or in another variant, all components are introduced concurrently. Embodiments lying between these two variants are of course possible and may be advantageous.

When adding the educts and catalysts it is possible to bring the various components into contact with each other either upstream of or in the reactor, as desired. Thus it is possible, for example, to effect previous formation of a complex of the boron compound and the chlorinating catalyst (eg the BF₃-β-picoline complex). Furthermore it is possible to cause previous reaction between the chlorinating catalyst and the chlorinating agent (eg Vilsmeier salt of N,N-dialkyl formamide and phosgene or thionyl chloride).

The process of the invention can be carried out batchwise or continuously.

a) Batchwise Mode

When manufacturing in batchwise mode, the reaction mixture containing the lactone (II), the chlorinating catalyst, the boron compound and optionally a solvent is generally placed in a reactor, for example an agitated tank, as the initial batch and mixed intensely. Then the desired amount of liquid or gaseous chlorinating agent is added at the desired temperature and pressure. After adding the chlorinating agent the reaction solution is allowed to continue reacting over a period ranging from a few minutes to a few hours. This subsequent reaction can take place in the reactor or in a vessel down-stream thereof.

In a special variant of the batchwise mode the liquid chlorinating agent (eg thionyl chloride) can be used as initial batch, optionally together with the chlorinating catalyst and/or the boron compound and/or a solvent. The lactone (II) is then, optionally together with the chlorinating catalyst and/or the boron compound and/or a solvent, added at the desired temperature and pressure over a given period of time.

b) Continuous Mode

Reactors that are suitable for the continuous process are for example stirred tanks, cascades of stirred-tank reactors or counter-current reaction towers. On starting the continuous process generally a solvent (eg the same chlorocarboxylic acid chloride as that to be produced), the chlorinating catalyst and the boron compound are placed in the reactor and the system is heated to the desired temperature, after which the liquid or gaseous chlorinating agent is added. Then, parallel to the continuous feed of chlorinating agent, there is started a continuous introduction of lactone (II), which generally contains further chlorinating catalyst and further boron compound and is optionally dissolved in a solvent. After the reactor contents have been converted to chlorocarboxylic chloride, the feed rates of lactone (II) and the chlorinating agent are adjusted such that both of these components are introduced in substantially equimolar amounts. Reaction mixture is removed from the reactor, for example through a riser or overflow, at a rate corresponding to the feed rate. Preferably, the reaction solution is fed to another vessel for further reaction.

It is then generally advantageous to expel (“strip”) unconverted chlorinating agent from the reaction solution, for example by passing in a gas which is chemically inert to the reaction solution, such as nitrogen.

Unconverted chlorinating agent which for example escapes from the reactor during the synthesis stage and/or is expelled by subsequent stripping, can advantageously be collected and reused. Suitable receivers are for example cold traps, in which the chlorinating agent condenses.

The reaction solution leaving the reaction of lactone (II) and the chlorinating agent can be worked up by conventional methods. Preference is given to purification by distillation, optional stripping being carried out upstream of or in the distillation column.

It is possible and may be advantageous to partially or completely recycle the bottoms obtained from purification by distillation and containing, inter alia, the chlorinating catalyst and the boron compound. Of course, another workup of the bottoms, for example distillation, to separate the chlorinating catalyst and/or the boron compound, can take place prior to said recycling operation. If the process is carried out with recycling of the chlorinating catalyst and/or boron compound, it is of advantage to recycle only a portion thereof, for the removal of possible by-products, and to replace the other portion by fresh catalysts.

In a general embodiment of the batchwise synthesis of chlorocarboxylic chlorides (I), all of the appropriate lactone (II), the (preferably liquid) chlorinating catalyst, the boron compound and, optionally, a solvent (eg the same chlorocarboxylic acid chloride as that to be produced) are placed in a stirred tank. The reaction system is then heated to the desired temperature and liquid and/or gaseous phosgene or liquid thionyl chloride is introduced continuously under ambient pressure with continued vigorous agitation. The resulting gaseous coupling products carbon dioxide or sulfur dioxide and also hydrogen chloride are removed. After the desired amount of chlorinating agent has been fed in, the reaction solution is left for a while at the controlled temperature, with continued agitation, for further reaction. During this subsequent reaction, chlorinating agent still present in the reaction solution reacts with the remaining lactone (II). In order to strip all or some of the excess chlorinating agent and its reaction products carbon dioxide or sulfur dioxide and hydrogen chloride, from the reaction solution, it is possible to pass through inert gas, with vigorous stirring. The resulting reaction solution is then passed on to the workup stage. Generally, workup is carried out by distillation, optionally in vacuo. In the case of high-molecular chlorocarboxylic chlorides, other purifying processes are possible, such as crystallization.

In a general embodiment for the continuous preparation of chlorocarboxylic chlorides (I) a solvent (eg the same chlorocarboxylic acid chloride as that to be produced), the chlorinating catalyst and the boron compound are placed in the reactor, eg a stirred tank, and the system is heated to the desired temperature and liquid or gaseous chlorinating agent is added. Then, parallel to the continuous feed of chlorinating agent, there is started a continuous introduction of lactone (II), which generally contains further chlorinating catalyst and further boron compound and is optionally dissolved in a solvent. After the reactor contents have been converted to chlorocarboxylic chloride, the feed rates of lactone (II) and the chlorinating agent are adjusted such that both of these components are introduced in substantially equimolar amounts. Reaction mixture is removed from the reactor, for example through a riser or overflow, at a rate corresponding to the feed rate. The removed reaction solution is collected in a vessel down-stream of the reactor, for example a stirred tank, for subsequent reaction. When the said downstream vessel is filled with said effluent, the overflow is optionally freed from the coupling products carbon dioxide and hydrogen chloride as described above and then passed on for workup. Workup can be carried out by distillation, for example.

The process of the invention allows for the preparation of chlorocarboxylic chlorides by reaction of the corresponding lactones with a chlorinating agent, and produces the chlorocarboxylic chlorides in a high yield and high state of purity and no longer suffers from the drawback of having to additionally feed in hydrogen chloride gas. During workup, the chlorocarboxylic chlorides can be readily separated from the boron compounds added in accordance with the invention.

### EXAMPLES

Experimental Setup

The experimental setup comprises a glass vessel having a capacity of 1L and equipped with a double-walled jacket and a stirrer, thermostatic control means, an inlet pipe for the gaseous or liquid chlorinating agent and a two-membered cascade of condensers. The two-membered cascade of condensers comprises a jacketed coil condenser, which is kept at −10° C., and a carbon dioxide condenser, which is kept at −78° C. The experiments were carried out under ambient pressure.

### Example 1 (Invention)

200 g (2.0 mol) of δ-valerolactone, 9.3 g (0.1 mol) of β-picoline (3-methylpyridine) and 3.1 g (0.05 mol) of boric acid were used as initial batch in the glass vessel having a double-walled jacket. A total of 229 g (2.32 mol) of gaseous phosgene were introduced at from 1440 to 148° C. over a period of 5 hours with vigorous stirring. The system was then left for a further hour without phosgene feed for subsequent reaction. After stripping off the remaining, unconverted phosgene with nitrogen a crude effluent weighing 310 g was obtained. The crude effluent was fractionally distilled at from 70° to 75° C. and under a pressure of 0.7 kPa absolute (7 mbar absolute). There were isolated 255 g of 5-chlorovaleric chloride having a purity of >98 GC-areal %. This corresponds to a yield of 82%.

### Example 2 (Invention)

172 g (2.0 mol) of γ-butyrolactone, 9.3 g (0.1 mol) of β-picoline (3-methylpyridine) and 3.1 g (0.05 mol) of boric acid were used as initial batch in the glass vessel having a double-walled jacket and heated to 140° C. A total of 242 g (2.45 mol) of gaseous phosgene were introduced at from 140° to 147° C. over a period of 4 hours and 15 minutes with vigorous stirring. The system was then left for a further hour without phosgene feed for subsequent reaction. After stripping off the remaining, unconverted phosgene with nitrogen at 100° C. a crude effluent weighing 289 g was obtained. The crude effluent contained 93.6 GC-areal % of 4-chlorobutyric chloride.

### Example 3 (Invention)

172 g (2 mol) of γ-butyrolactone, 34.8 g (0.1 mol) of Cyanex® 923 (commercial product sold by Cytec Industries and comprising a mixture of various trialkyl phosphine oxides having an average molecular weight of 348 g/mol) and 3.1 g (0.05 mol) of boric acid were used as initial batch in the glass vessel having a double-walled jacket. A total of 251 g (2.54 mol) of gaseous phosgene were introduced at from 144° to 148° C. over a period of 5 hours and 20 minutes with vigorous stirring. The system was then left for a further hour without phosgene feed for subsequent reaction. After stripping off the remaining, unconverted phosgene with nitrogen at 100° C. over a period of 7 hours a crude effluent weighing 314 g was obtained. The crude effluent was fractionally distilled at 87° C. under a pressure of 5.1 kPa absolute (51 mbar absolute). There were isolated 242 g of 4-chlorobutyric chloride having a purity of >99 GC-areal %. This corresponds to a yield of 86%.

### Example 4 (Invention)

200 g (2.0 mol) of δ-valerolactone, 9.3 g (0.1 mol) of β-picoline (3-methylpyridine) and 5.2 g (0.05 mol) of trimethyl borate were used as initial batch in the glass vessel having a double-walled jacket and heated to 140° C. A total of 242 g (2.45 mol) of gaseous phosgene were introduced at from 140° to 146° C. with vigorous stirring. The system was then left for a further hour without phosgene feed for subsequent reaction. After stripping off the remaining, unconverted phosgene with nitrogen at 100° C. a crude effluent weighing 318 g was obtained. The crude effluent was fractionally distilled at from 75° to 77° C. under a pressure of 0.9 kPa absolute (9 mbar absolute). Following first runnings weighing 10 g, which already contained 96.6 GC-areal % of 5-chlorovaleric chloride, there was isolated a pure fraction weighing 256 g. It contained 98.2 GC-areal % of 5-chlorovaleric chloride. The total yield following distillation was 85%.

### Example 5 (Invention)

10 g (0.1 mol) of δ-valerolactone, 1.14 g (0.006 mol) of benzyl-trimethylammonium chloride and 0.31 g (0.005 mol) of boric acid were used as initial batch in the glass vessel having a double-walled jacket. A total of 15.5 g (0.13 mol) of liquid thionyl chloride were introduced at from 120° to 125° C. over a period of 7 hours with vigorous stirring. The system was then left for a further hour without thionyl chloride feed for subsequent reaction. The effluent contained 70 GC-areal % of 5-chlorovaleric chloride and 7 GC-areal % of unconverted δ-valerolactone.

### Example 6 (Comparative Example)

192 g (2.23 mol) of γ-butyrolactone and 2 g (0.025 mol) of pyridine were used as initial batch in the glass vessel having a double-walled jacket and heated to 120° C. A total of 60 g (0.61 mol) of gaseous phosgene were introduced at from 120° to 124° C. over a period of 8 hours with vigorous stirring. After stripping off the remaining, unconverted phosgene with nitrogen the crude effluent was fractionally distilled. The first fraction weighing 76 g contained 21.6 GC-areal % of 4-chlorobutyric chloride, the second fraction weighing 110 g contained 2.6 GC-areal % of 4-chlorobutyric chloride. This corresponds to a total yield of 6%.

Comparative Example 6 shows that in the absence of boron compounds and without the introduction of hydrogen chloride only insufficient yield can be attained.

We claim:

1. A process for preparing chlorocarbonyl chlorides of the formula (I) 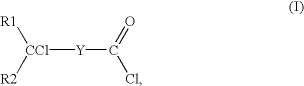
in which
R¹ and R² independently of one another
are a hydrogen atom, a carbon-containing organic radical, a halogen, a nitro or a cyano group,
and Y
is an alkylene chain having 1 to 10 carbon atoms in the chain, which is unsubstituted or substituted by carbon-containing organic radicals, halogen, nitro and/or cyano groups, where the alkylene chain may be interrupted by an ether, a thioether, a tertiary amino or a keto group,
where the carbon-containing organic radicals of Y and/or R¹ and/or R² may be attached to one another forming a nonaromatic system, by reacting a lactone of the formula (ii) 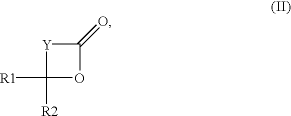
in which R¹, R² and Y are as defined above, with a chlorinating agent in the presence of a chlorination catalyst, which comprises carrying out the reaction in the presence of boron oxide, oxoboric acids, salts of the oxoboric acids or mixtures thereof.

2. A process as claimed in claim 1, wherein boric acid is used.

3. A process as claimed in claim 1 or 2, wherein the boron compound is employed in a concentration of 0.1-20 mol %, based on the lactone (II).

4. A process as claimed in any of claims 1 to 3, wherein the chlorinating agent used is phosgene, diphosgene, triphosgene or thionyl chloride.

5. A process as claimed in any of claims 1 to 4, wherein the chlorination catalyst used is a urea compound, a phosphine oxide, a pyridinium compound or a mixture thereof.

6. A process as claimed in claim 5, wherein the chlorination catalyst used is 3-methylpyridine, triphenylphosphine oxide and/or trialkylphosphine oxide.

7. A process as claimed in any of claims 1 to 6, wherein the chlorination catalyst is employed in a concentration of from 0.1 to 20 mol %, based on the lactone (II).

8. A process as claimed in any of claims 1 to 7, wherein the chlorination catalyst and the boron compound are employed in the form of a complex of the two components.

9. A process as claimed in any of claims 1 to 8, wherein the reaction is carried out at a temperature of 50-200° C. and an absolute pressure of 0.01-5 MPa.

10. A process as claimed in any of claims 1 to 9, wherein the lactone (II) used is γ-butyrolactone, δ-valerolactone or ε-caprolactone.
